(19) United States
(12) Patent Application Publication
(10) Pub. No.: US 2019/0295732 A1
(43) Pub. Date: Sep. 26, 2019

(54) APPARATUS FOR PREDICTING OUTBREAK OF AVIAN INFLUENZA

(71) Applicant: G-LAND, Gyeonggi-do (KR)

(72) Inventor: Tai-Jin KIM, Gyeonggi-do (KR)

(21) Appl. No.: 16/044,740

(22) Filed: Jul. 25, 2018

(30) Foreign Application Priority Data
Mar. 21, 2018 (KR) 10-2018-0032774

(51) Int. Cl. G16H 50/80

(52) U.S. Cl. CPC G16H 50/80; G06F 9/06

(57) ABSTRACT

There is provided a prediction apparatus. The prediction apparatus includes a storage unit storing information on the amount of CO₂ measured in the atmosphere and the number of sunspots and a prediction unit configured to determine possibility of an AI outbreak while considering the amount of CO₂ measured in the atmosphere or the number of sunspots that are stored in the storage unit.

### CROSS-REFERENCE TO RELATED APPLICATIONS

This application is based on and claims priority from Korean Patent Application No. 10-2018-0032774, filed on Mar. 21, 2018, the disclosure of which is incorporated herein in its entirety by reference.

### TECHNICAL FIELD

The present disclosure relates generally to an apparatus for predicting an outbreak of avian influenza (AI).

### BACKGROUND

Avian influenza viruses (AIV) cause widespread morbidity and mortality in a broad range of hosts such as birds, swine, companion animals, marine animals, and humans. Influenza viruses are roughly spherical (120 nm) with glycoprotein spikes on the surface and genome consisting of eight RNA fragments that encode 10 proteins. The hemagglutinin (HA), neuraminidase (NA) and matrix (M2) proteins are embedded in the envelope lipid bilayer derived from the host cell. Since there are currently 18 HA and 11 NA subtypes and thus 198 combinations with 4 strains of A, B, C and D, blocking the cell-to-cell spread of AIV is difficult to prevent in advance, regardless of a vaccine.

### SUMMARY

In view of the above, aspects of the present disclosure provide an apparatus for predicting an outbreak of AVIAN INFLUENZA.

However, aspects of the present disclosure are not restricted to those set forth herein. The above and other aspects of the present disclosure will become more apparent to those skilled in the art to which the present disclosure pertains by referencing the detailed description of the present disclosure given below.

In accordance with an embodiment of the present disclosure, there is provided a prediction apparatus. The prediction apparatus includes a storage unit that stores information on the amount of CO₂ measured in the atmosphere and the number of sunspots, and a prediction unit configured to determine the possibility of an AI outbreak while considering the amount of CO₂ measured in the atmosphere or the number of sunspots that are stored in the storage unit.

Further, the prediction unit may be configured to predict that the possibility of an AI outbreak is higher when the amount of CO₂ measured in the atmosphere is relatively large than when the amount of CO₂ measured in the atmosphere is relatively small.

Further, the prediction unit may be configured to predict that the possibility of an AI outbreak is higher when the number of sunspots is relatively small than when the number of sunspots is relatively large.

Further, the storage unit may store information on the thickness of the ozone layer in polar regions, and the prediction unit may be configured to predict the possibility of an AI outbreak while further considering the information on the thickness of the ozone layer in the polar regions stored in the storage unit.

Further, the storage unit may store the direction of the wind, the frequency of desert dust incidences, the cultivation area of crops and the migratory routes of migratory birds on a region basis, and the prediction unit may be configured to predict the possibility of an AI outbreak while further considering the direction of wind, the frequency of desert dust incidences, the cultivation area of crops and the migratory route of migratory birds that are stored in the storage unit on a region basis.

### BRIEF DESCRIPTION OF THE DRAWINGS

The objects and features of the disclosure will become apparent from the following description of embodiments, given in conjunction with the accompanying drawings, in which:

FIG. 1 is a block diagram showing a configuration of an apparatus for predicting an outbreak of avian influenza (AI) according to an embodiment.

FIG. 2 is the first flow chart showing a process of a method for predicting the possibility of an AI outbreak according to an embodiment.

FIG. 3 is the second flow chart showing a process of a method for predicting the possibility of an AI outbreak according to an embodiment.

FIG. 4 is the third flow chart showing a process of a method for predicting the possibility of an AI outbreak according to an embodiment.

FIG. 5 is a table showing unfavorable conditions and favorable conditions according to an embodiment.

FIG. 6 shows an average daily sunspot area (or the number of sunspot) as a function of time between years of 1870 and 2020.

FIG. 7 shows that the linear relationship of the year of AIV outbreak was strongly correlated with the year of minimal average daily sunspot area.

FIG. 8 shows the prediction of sunspot number between years of 2000 and 2018.

FIGS. 9 and 10 show carbon dioxide emissions from fossil fuel burning between years of 1850 and 2016.

### DETAILED DESCRIPTION

The advantages and features of the present disclosure and the methods of accomplishing these will be clearly understood from the following description taken in conjunction with the accompanying drawings. However, embodiments are not limited to those embodiments described, as embodiments may be implemented in various forms. It should be noted that the present embodiments are provided to make a full disclosure and also to allow those skilled in the art to know the full range of the embodiments. Therefore, the embodiments are to be defined only by the scope of the appended claims.

In describing the embodiments of the present disclosure, if it is determined that detailed description of related known components or functions unnecessarily obscures the gist of the present disclosure, the detailed description thereof will be omitted. Further, the terminologies to be described below are defined in consideration of functions of the embodiments of the present disclosure and may vary depending on a user's or an operator's intention or practice. Accordingly, the definition thereof may be made on a basis of the content throughout the specification.

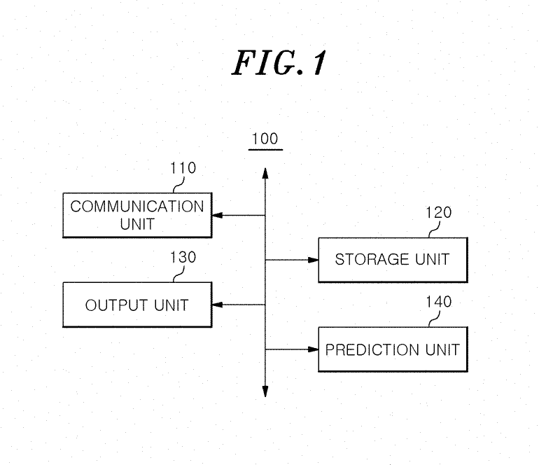

FIG. 1 is a block diagram showing a configuration of an apparatus for predicting an outbreak of avian influenza (AI) according to an embodiment. Referring to FIG. 1, a prediction apparatus 100 includes a communication unit 110, a storage unit 120, an output unit 130 and a prediction unit 140. The configuration of the prediction apparatus 100 shown in FIG. 1 is merely an example. Therefore, the prediction apparatus 100 may further include a component that is not shown in FIG. 1 or may not include at least one of the components shown in FIG. 1 depending on the embodiment.

The communication unit 110 includes a wired or a wireless communication module. The prediction apparatus 100 can communicate with various external servers via the communication unit 110. For example, the prediction apparatus 100 can be connected to a server in which data collected by a satellite is stored or a server that provides global climate and weather conditions in real time, and can receive data from those servers.

The storage unit 120 includes a memory for storing data. The storage unit 120 can store data used for prediction performed by the prediction unit 140, which will be described later. For example, the storage unit 120 can store the following data. However, the data stored in the storage unit 120 is not limited to the following data.
(1) global CO₂ emission amount, the number of sunspots
(2) the thickness of an ozone layer, the amount of UV rays reaching the ground or the sea surface
(3) the temperature or humidity on the ground, rainfall, direction of wind, the frequency of desert dust incidences, the level of desert dust, the cultivation area of crops such as wheat or rice, the degree of melting of glaciers, the amount of algae, the amount of krill or zooplankton, the migratory route of migratory birds, seawater salinity

Here, each of the data (3) may be measured in each region per unit of time (predetermined period of time), and each of the data (1) and (2) may be measured in the polar regions per unit of time. Further, each of the data stored in the storage unit 120 can be updated.

The data (1) will be referred to as “independent variable” and the data (2) and (3) will be referred to as “dependent variable”. This is because the data (2) and (3) may be determined or changed by the data (1) or other factors. For example, when the CO₂ emission amount is increased, the thickness of the ozone layer in the polar regions may be decreased. Thus, the amount of UV radiation in the polar regions may be increased. When the amount of UV radiation in the polar regions is increased, the temperature of the ground may be increased. Accordingly, the glaciers may melt faster in the polar regions. As a result, the seawater salinity may be decreased.

The output unit 130 outputs the prediction result of the prediction unit 140 to be described later. The output unit 130 can be implemented by a display device.

The prediction unit 140 can be implemented by a memory for storing commands programmed to perform functions to be described later and a microprocessor for executing the commands stored in the memory.

Hereinafter, the prediction process performed by the prediction unit 140 based on the principle mentioned above will be described.

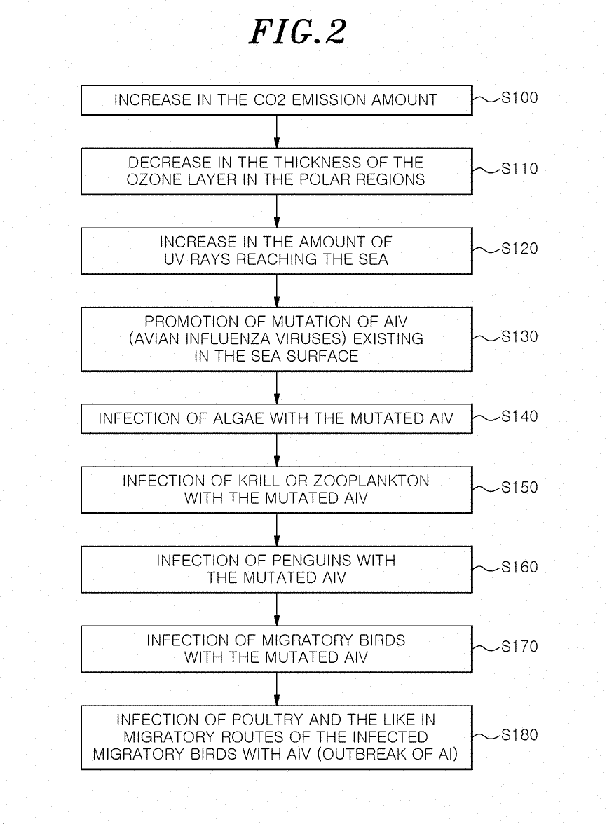

The prediction unit 140 can predict the possibility of an AI outbreak by using the data stored in the storage unit 120, i.e., one of the above-described data (1) to (3), as shown in FIGS. 2 to 4.

Referring to FIGS. 2 to 4, the prediction unit 140 can predict the possibility of an AI outbreak by using one of the above-described data (1) (independent variable).

For example, the prediction unit 140 can predict that the possibility of an AI outbreak is increased as the global CO₂ emission amount is increased or as the number of sunspots is decreased.

The prediction unit 140 can predict as described above by employing at least one of the following three predetermined prediction algorithms.

### <First Prediction Algorithm> (Shown in FIG. 2)

Increase in the CO₂ emission amount (S100)→decrease in the thickness of the ozone layer in the polar regions (S110)→increase in the amount of UV rays reaching the sea surface (S120)→promotion of mutation of viruses existing in the sea surface (S130)→infection of migratory birds and the like with the mutated virus transmitted via food chain (S140 to 5170)→infection of poultry and the like in migratory routes of the infected migratory birds with AIV (S180)

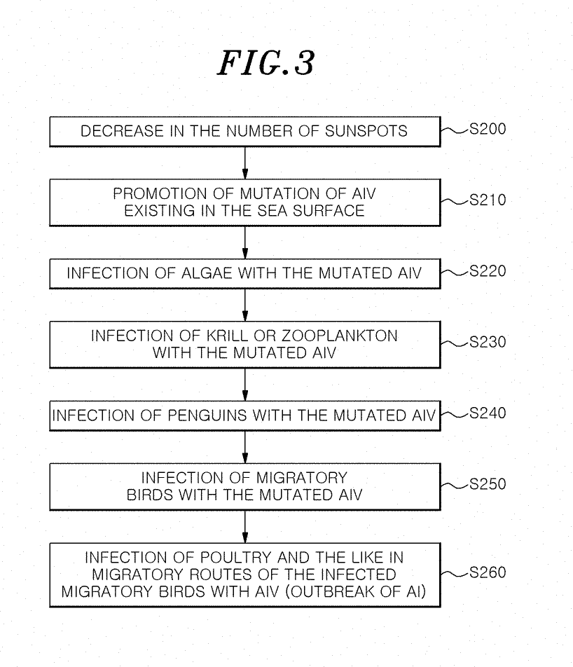

### <Second Prediction Algorithm> (Shown in FIG. 3)

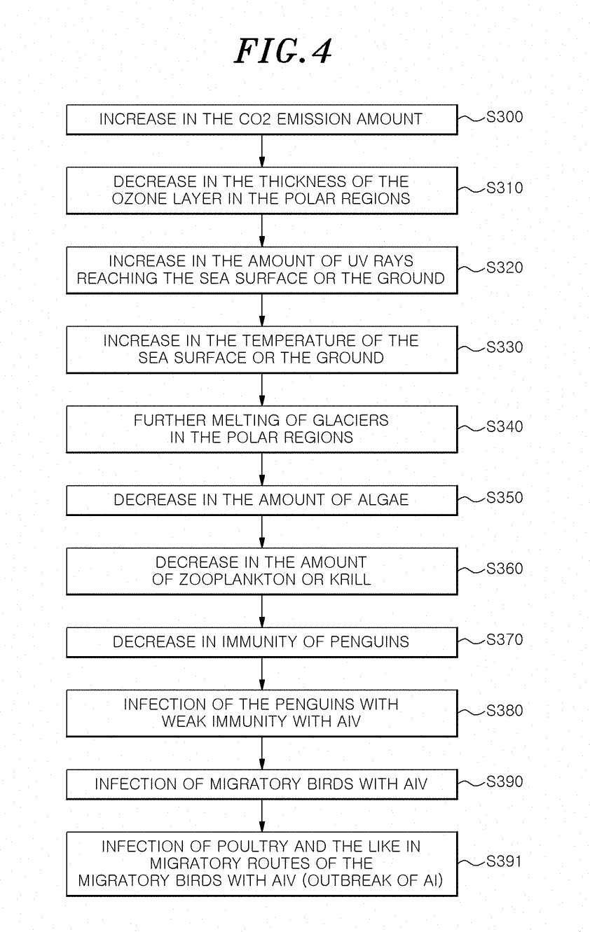

Decrease in the number of sunspots (S200)→promotion of mutation of viruses (S210)→infection of migratory birds and the like with the mutated virus transmitted via the food chain (S220 to 5250)→infection of poultry and the like in migratory routes of the infected migratory birds with AIV (S260) <Third Prediction Algorithm> (Shown in FIG. 4)

Increase in the CO₂ emission amount (S300)→decrease in the thickness of the ozone layer in the polar regions (S310)→increase in the amount of UV rays reaching the sea surface or the ground (S320)→increase in the temperature of the sea surface or the ground (S330)→further melting of glaciers in the polar regions (S340)→decrease in the amount of algae (S350)→decrease in the amount of zooplankton or krill (S360)→decrease in immunity of penguins eating zooplankton or krill due to the decrease in the amount of zooplankton or krill (S370)→infection of penguins with weak immunity with AIV (S380)→infection of migratory birds with AIV (S390)→infection of poultry and the like in migratory routes of the migratory birds with AIV (S391)

Hereinafter, the first prediction algorithm will be described in detail.

The increase in the CO₂ concentration may cause the greenhouse effect, which may result in climate changes. Due to the climate changes, O₃ constituting the ozone layer may be destroyed and, thus, the thickness of the ozone layer may be decreased. When the thickness of the ozone layer is decreased, the amount of UV rays reaching the sea surface may be increased. When the amount of UV rays reaching the sea surface is increased, mutation of viruses existing in the sea surfaces may be promoted. The mutated virus may be transferred to migratory birds via the food chain. Accordingly, the migratory birds may be infected with the mutated virus. At this time, the AI occurring in migratory birds by the mutated AIV may be a low pathogenic avian influenza (LPAI). As the migratory birds infected with the mutated AIV migrate along the migratory route, poultry in the migratory route of the migratory birds may be infected with the mutated AIV.

Next, the second prediction algorithm will be described in detail.

The decrease in the number of sunspots indicates that the amount of UV rays emitted from the sun may be decreased. Therefore, the decrease in the number of sunspots indicates that the amount of UV rays reaching the sea surface may be decreased. When the amount of UV rays reaching the sea surface becomes minimum, i.e., when the number of sunspots becomes minimum, the mutation of viruses existing in the sea surface can become maximum. The mutated virus can be transferred to migratory birds via the food chain. As a consequence, the migratory birds are infected with the mutated virus. At this time, the AI occurring in the migratory birds by the mutated AIV may be an LPAI. As the migratory birds infected with the mutated AIV migrate along the migratory routes, poultry in the migratory route of the migratory birds may be infected with the mutated AIV.

The prediction unit 140 predicts that the possibility in which the migratory birds are infected with AIV via the food chain is higher in the case of the mutated AIV than in the case of the non-mutated AIV. Therefore, the prediction unit 140 predicts that the possibility of an AI outbreak becomes higher as the CO₂ concentration is increased or as the number of sunspots is decreased, i.e., as the degree of mutation of AIV is increased.

Here, the food chain that is the route through which the mutated virus is transferred to the migratory birds will be described. The mutated virus may exist in algae, krill or zooplankton living in the sea. Therefore, fish and birds eating algae, krill or zooplankton may be infected with the mutated virus. The birds eating the mutated virus may be penguins. Penguins may be infected with the mutated virus, and migratory birds staying in the habitat of the infected penguins (polar regions) may be infected with the mutated virus.

Next, the third prediction algorithm will be described in detail.

The increase in the CO₂ concentration may cause the greenhouse effect, which may result in climate changes. Due to the climate changes, O₃ constituting the ozone layer may be destroyed and, thus, the thickness of the ozone layer may be decreased. When the thickness of the ozone layer is decreased, the amount of UV rays reaching the sea surface or the ground may be increased. When the amount of UV rays reaching the sea surface or the ground is increased, the temperature of the sea surface of the ground may be increased. Accordingly, more glaciers may be melted in the polar regions, and the melted glaciers flow to the ocean. As a result, the amount of algae can be reduced and, further, the amount of zooplankton or krill can be reduced. When the amount of zooplankton or krill is reduced, the immunity of penguins eating zooplankton or krill may become weak. Penguins with weak immunity may be easily infected with virus, which may lead to an increase in the possibility in which migratory birds staying in the habitat of the penguins are infected with virus. As a result, the possibility in which poultry and the like in the migratory route of the infected migratory birds are infected with AIV may be increased.

Further, the prediction unit 140 may predict the possibility of AI outbreak by using the above-described data (2) or (3) (dependent variable).

For example, the prediction unit 140 can acquire the information on the thickness of the ozone layer in the polar regions from the storage unit 130. The prediction unit 140 can predict that the possibility of an AI outbreak is increased as the thickness of the ozone layer in the polar regions is decreased. The prediction unit 140 can predict that the possibility of an AI outbreak is decreased as the thickness of the ozone layer is increased.

Further, the prediction unit 140 can predict the possibility of an AI outbreak as follows by using various data (dependent variable) in addition to the information on the thickness of the ozone layer in the polar regions.
The possibility of an AI outbreak is increased (decreased) as the frequency of desert dust incidences is increased (decreased).
The possibility of an AI outbreak is increased (decreased) as the amount of melted glaciers is increased (decreased).
The possibility of an AI outbreak is increased (decreased) as the seawater salinity is decreased (increased).
The possibility of an AI outbreak is increased (decreased) as the amounts of algae, krill or zooplankton is increased (decreased).

The prediction unit 140 predicts that the possibility of an AI outbreak is increased as the frequency of desert dust incidences is increased based on the following predetermined prediction algorithm.
desert dust contains various proteins and various components (Fe, Cd, Mg, Mo, Co, V and Ni) required to activate virus. Therefore, as the frequency of desert dust incidences is increased, the possibility of an AI outbreak is increased.

The prediction unit 140 can predict the possibility of an AI outbreak by combining at least two of the data (1) to (3) (i.e., while considering at least two of the data (1) to (3)). In that case, the weight may be set in advance for each data. The weight is a value that reflects the effect of each data on the possibility of an AI outbreak.

Up to now, the process in which the prediction unit 140 predicts the possibility of an AI outbreak has been described. Hereinafter, a process in which the prediction unit 140 predicts whether the AI occurring in each region is LPAI or highly pathogenic avian influenza (HPAI) will be described.

The prediction unit 140 can predict whether the AI occurring in each region is LPAI or HPAI by using at least one of the data (1) to (3) stored in the storage unit 120.

At this time, the weights may be set in advance for each of the data. Therefore, the prediction unit 140 considers the weights in the case of using a combination of at least two of the data (1) to (3).

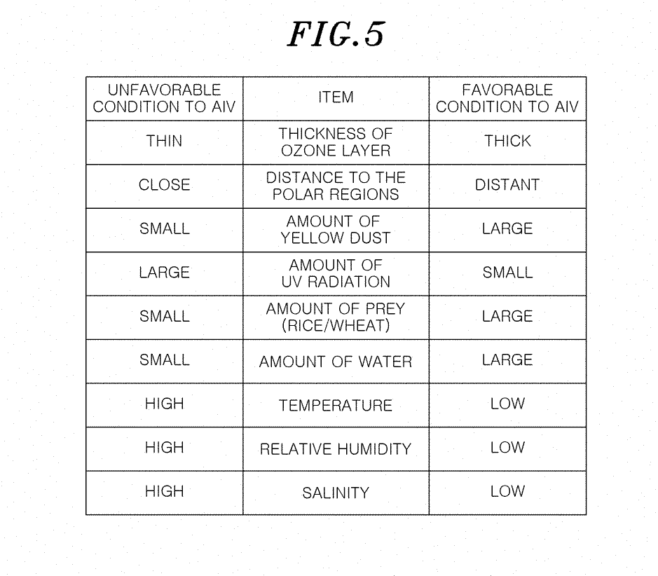

Specifically, LPAI occurs in migratory birds as described above. However, even if the migratory birds are infected with LPAI, poultry in each region may be infected with LPAI or HPAI depending on the conditions of each region. Hereinafter, the conditions in which LPAI occurs in poultry in each region will be referred to as “unfavorable conditions”, and the conditions in which HPAI occurs in poultry in each region will be referred to as “favorable conditions”. The unfavorable conditions and the favorable conditions are shown in FIG. 5. Hereinafter, the unfavorable conditions and the favorable conditions will be described with reference to FIG. 5.

The unfavorable conditions to AIV include the following conditions but are not limited thereto.

relatively thin ozone layer

relatively close to the polar regions

relatively not enough desert dust

relatively high UV radiation

relatively not enough prey (rice/wheat)

relatively not enough water

relatively high temperature

relatively high humidity (relative humidity)

relatively high salinity

The favorable conditions to AIV include the following conditions but are not limited thereto.

relatively thick ozone layer

relatively far from the polar regions

relatively enough desert dust

relatively low UV radiation

relatively enough prey (rice/wheat)

relatively enough water

relatively low temperature

relatively low humidity (relative humidity)

relatively low salinity

The favorable conditions to AIV and the unfavorable conditions to AIV have corresponding items. The storage unit 120 stores data on whether each region satisfies the favorable conditions or the unfavorable conditions. The prediction unit 140 can acquire the stored data from the storage unit 120 and predict whether LPAI or HPAI has occurred in each region.

For example, it is assumed that there are regions A and B. According to the data stored in the storage unit 120, the two regions have the same conditions for the thickness of the ozone layer, the distance to the polar regions, the amount of desert dust, the amount of UV radiation, the amount of prey (rice or wheat), the amount of water, the temperature, and the humidity. However, the salinity is higher in region A than in region B. In that case, the prediction unit 140 can predict that the possibility of LPAI outbreak is higher in region A and the possibility of HPAI outbreak is higher in region B.

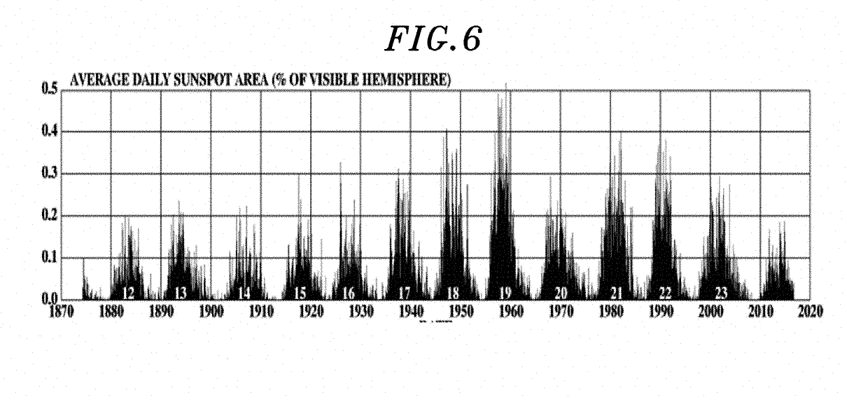

Meanwhile, FIG. 6 shows an average daily sunspot area (or the number of sunspot) as a function of time between years of 1870 and 2020.

Referring to FIG. 6, the average area of the Antarctic ozone hole was the smallest in 1995 and the 10.7 cm solar flux index showed the minimum. Besides, the minimum sunspot number is proportional to the 10.7 cm solar flux. Therefore, there was a strong HPAI H5N1 in Hong Kong during 1995-1996. It can be postulated that there can be an AIV outbreak when the sunspot number or the 10.7 cm solar flux are minimal.

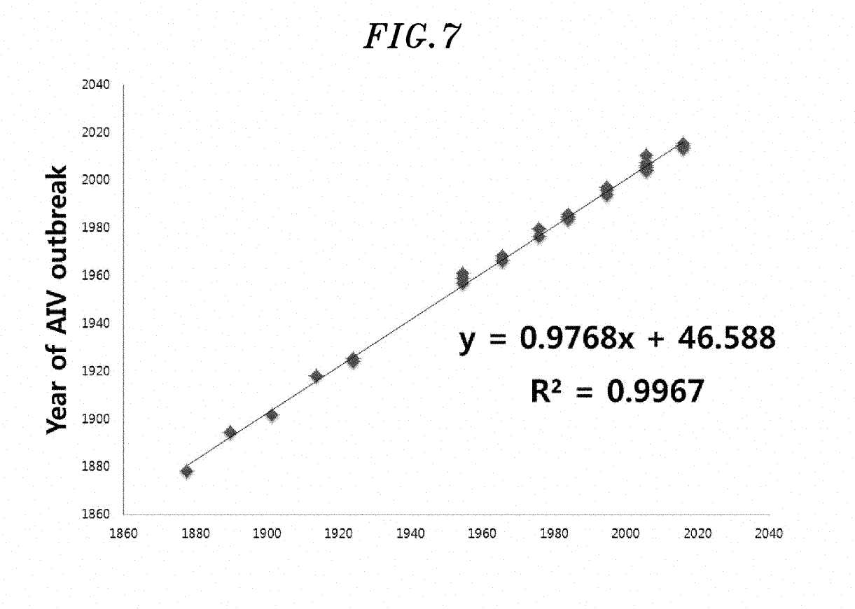

FIG. 7 shows that the linear relationship of the year of AIV outbreak was strongly correlated (R2=0.9967) with the year of minimal average daily sunspot area. The timing of AIV outbreak is expected in the period of the minimum sunspot number (minimal average daily sunspot area).

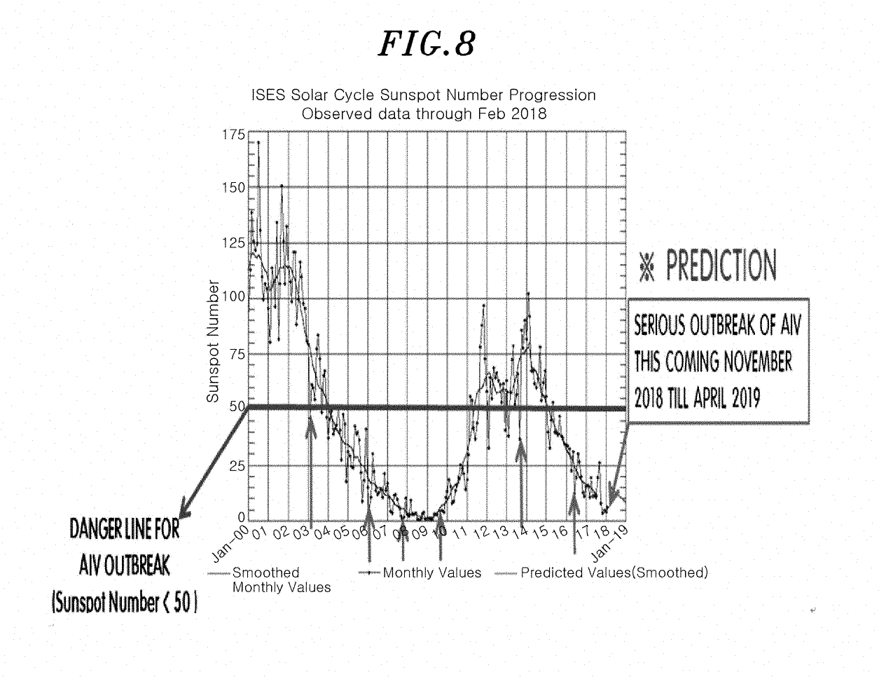

FIG. 8 shows the prediction of sunspot number between years of 2000 and 2018. Referring to FIG. 8, there were 2% abrupt rise of global CO2 emissions in 2017 as well as reaching the minimum sunspot number in the end of 2018. Outbreaks of AIV with the highest degree of damage are thus expected during November 2018 till April 2019 in North America, East Asia, China, South Korea, Japan, west Africa, and Europe. AIV were occurred when sunspot numbers were below 50, as indicated in arrows in FIG. 8.

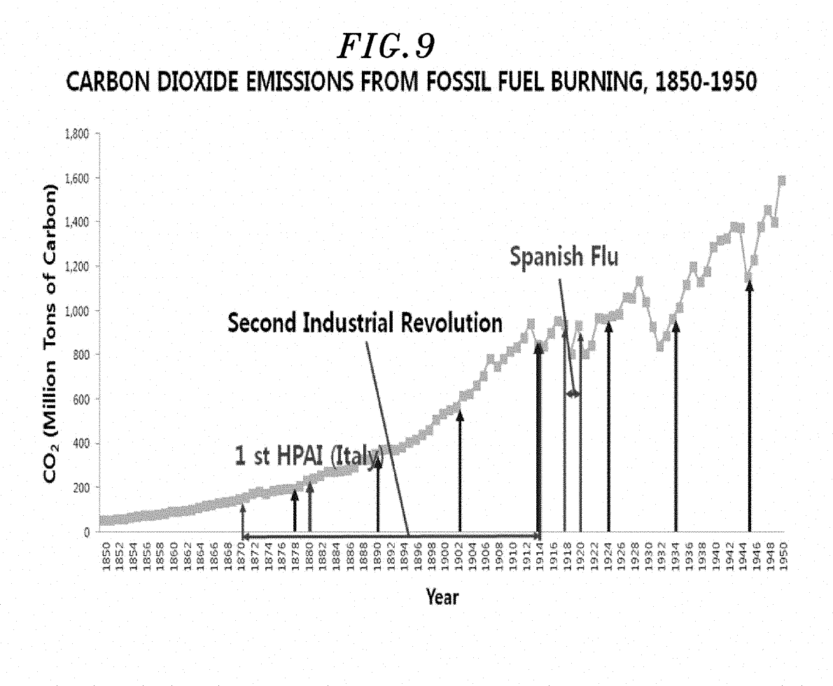

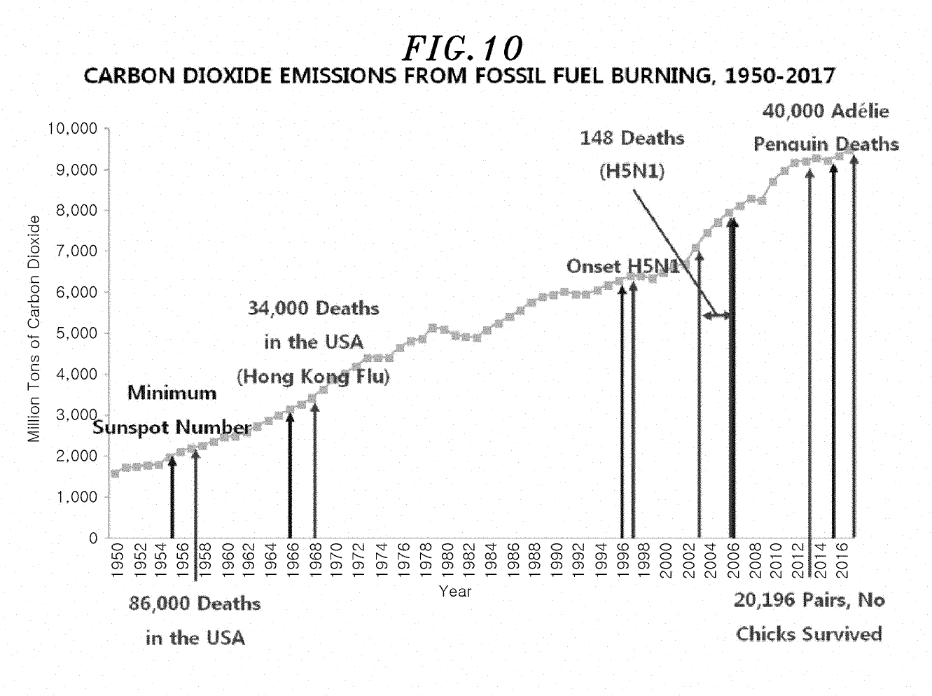

FIGS. 9 and 10 show carbon dioxide emissions from fossil fuel burning between years of 1850 and 2016. Referring to FIGS. 9 and 10, the Second Industrial Revolution is generally dated between 1870 and 1914 while the first highly HPAI occurred in Italy in 1878 and the 1918 pandemic Spanish flu during January 1918 to December 1920 resulted in the deaths of 50 to 100 million as one of the deadliest natural disasters in human history. It is postulated that global atmospheric CO2 increase since the start of The Second Industrial Revolution might ultimately induce AIV outbreaks of HPAI H1N1 in the Continents. This is a remarkable discovery to find out the initiative relation between AIV outbreak and the climate change induced by global atmospheric CO2 increases.

As described above, in accordance with one embodiment, the possibility of an AI outbreak can be predicted by global atmospheric CO₂ increases. Since the data used for the prediction can be received or updated in real time from outside sources such as satellites, the above-described prediction, i.e., the prediction of AI, can be made in real time.

As described above, those skilled in the art will understand that the present disclosure can be implemented in other forms without changing the technical idea or essential features thereof. Therefore, it should be understood that the above-described embodiments are merely examples, and are not intended to limit the present disclosure. The scope of the present disclosure is defined by the accompanying claims rather than the detailed description, and the meaning and scope of the claims and all changes and modifications derived from the equivalents thereof should be interpreted as being included in the scope of the present disclosure.

What is claimed is:

1. A prediction apparatus comprising:
a storage unit storing information on the amount of CO₂ measured in the atmosphere and the number of sunspots; and
a prediction unit configured to determine a possibility of an AI outbreak while considering the amount of CO₂ measured in the atmosphere or the number of sunspots that are stored in the storage unit.

2. The prediction apparatus of claim 1, wherein the prediction unit is configured to predict that the possibility of the AI outbreak is higher when the amount of CO₂ measured in the atmosphere is relatively large than when the amount of CO₂ measured in the atmosphere is relatively small.

3. The prediction apparatus of claim 1, wherein the prediction unit is configured to predict that the possibility of the AI outbreak is higher when the number of sunspots is relatively small than when the number of sunspots is relatively large.

4. The prediction apparatus of claim 1, wherein the storage unit further stores information on a thickness of an ozone layer in polar regions, and
the prediction unit is configured to predict the possibility of the AI outbreak while further considering the information on the thickness of the ozone layer in the polar regions that is stored in the storage unit.

5. The prediction apparatus of claim 1, wherein the storage unit stores a direction of wind, a frequency of desert dust incidences, a cultivation area of crops and a migratory route of migratory birds on a region basis, and
the prediction unit is configured to predict the possibility of the AI outbreak while further considering the direction of wind, the frequency of desert dust incidences, the cultivation area of crops and the migratory route of migratory birds that are stored in the storage unit on a region basis.
